(19) United States
(12) Patent Application Publication
(10) Pub. No.: US 2019/0008969 A1
(43) Pub. Date: Jan. 10, 2019

(54) COMPOSITIONS FOR DRUG DELIVERY

(71) Applicants: Tioga Research, Inc., San Diego, CA (US); Nivagen Pharmaceuticals, Inc., Sacramento, CA (US)

(72) Inventors: Edward Thomas Kisak, San Diego, CA (US); John Michael Newsam, La Jolla, CA (US); Avadhesh Kushwaha, San Diego, CA (US)

(21) Appl. No.: 15/643,106

(22) Filed: Jul. 6, 2017

(51) Int. Cl. A61K 47/28; A61K 47/14; A61K 47/10; C07C 31/10

(52) U.S. Cl. CPC A61K 47/28; A61K 47/14; C11C 3/04; C07C 31/10; A61K 47/10

(57) ABSTRACT

The present disclosure relates generally to compositions and formulations of active agents for improved delivery of the active agents, methods of making such compositions or formulations, and methods of use thereof. Also described herein are compositions comprising isopropyl palmitate and formulations thereof for drug delivery of an active agent.

### FIELD

The present disclosure relates to compositions and formulations of active agents, such as corticosteroids, for topical applications. Such compositions and formulations are useful in the treatment of skin diseases.

### BACKGROUND

Dermatological conditions, such as psoriasis and atopic eczema, are prevalent, and they can have a very negative impact on the quality of life of sufferers. Human inflammatory immune reactions that impact the skin, such as those implicated in psoriasis and atopic eczema, are regulated via endogenous glucocorticoids (the name deriving from the construct glucose+cortex+steroid), such as cortisol (hydrocortisone). Over the more than 60 years since topical hydrocortisone was first used to successfully treat atopic dermatitis in 1952, the slate of approved topical corticosteroid formulations has expanded dramatically.

Glucocorticoids bind to the glucocorticoid receptor (“GCR”), which is present in nearly all vertebrate cells. They act by inhibiting genes that code for the cytokine IL-2, as well as IL-1, IL-3, IL-4, IL-5, IL-6, IL-8 and TNT-alpha; reduced cytokine production limits T cell proliferation. Glucocorticoids also suppress humoral immunity, causing B cells to express lower amounts of IL-2 and IL-2 receptors, diminishing B cell clonal expansion and antibody synthesis. Reduced IL-2 production also leads to fewer T lymphocyte cells being activated. Topical corticosteroids thus have anti-inflammatory, antiproliferative and atrophogenic effects in the skin, as well as, to some degree, the systemic effects seen with oral corticosteroids.

Desoximetasone ((8S,9S,10S,11S,13S,14S,16R,17S)-9-fluoro-11-hydroxy-17-(2-hydroxyacetyl)-10,13,16-trirmethyl-7,8,11,12,14,15,16,17-octahydro-6H-cyclopenta[a]phenanthren-3-one) is a Group 11, high potency corticosteroid (“UHPS”) that is amongst the less well studied corticosteroids. Phase III clinical study results showed that desoximetasone was efficacious and safe in treating psoriasis in the sampled population when dosed in a spray format at 0.25 wt %.

Although large numbers of topical glucocorticoid products are currently available on the market, either under prescription or over the counter (“OTC”), no product has universal effectiveness. Rather, a patient must work to select a product appropriate for the state of their disease, by balancing (i) glucocorticoid potency, (ii) the extent to which their specific disease responds to the given glucocorticoid, (iii) duration of intended application, (iv) extent of potential depression of the hypothalamic-pituitary-adrenal (“HPA”) axis, (v) esthetics aspects, (vi) availability, and (vii) cost. Certain of these aspects that determine the benefit of a given topical glucocorticoid product for a given patient at a given moment in time reflect properties, not of the glucocorticoid active, but of the formulation and its manner of administration.

There, therefore, remains a continuing need for compositions and formulations suitable for administration of corticosteroids to the skin of a subject in need thereof.

Further, to be useful to patients in practice, a topical pharmaceutical formulation must satisfy, simultaneously, multiple criteria. Even for an active pharmaceutical ingredient (“API”) at a specified strength in a topical formulation, there is a need for alternative formulations of the same API and at the same strength that comprise different excipients. There is, therefore, a need for a method that can be used to identify topical formulations that differ in composition from a target formulation yet that exhibit properties determining pharmaceutical utility that are similar to those of the target formulation.

### SUMMARY

The present disclosure provides for compositions and formulations of active agents for improved delivery of the active agent, methods of making such compositions or formulations, and methods of use thereof, as well as methods for identifying formulations that evidence properties similar to those of a target formulation.

Some embodiments provide for a topical composition comprising an active agent and an ester of a C₂₋₄ alcohol and a fatty acid, wherein an effective amount of the active agent is delivered to the dermis or epidermis.

Some embodiments provide for a topical composition comprising:

(a) about 0.01 wt % to about 2.5 wt % of a corticosteroid;

(b) an ester of a C₂₋₄ alcohol and a fatty acid;

(c) about 10 wt % to about 80 wt % of a C₂₋₄ alcohol;

(d) about 3 wt % to about 75 wt % of an oleaginous vehicle selected from mineral oil, soybean oil, caprylic/capric triglycerides, and a combination thereof; and

(e) about 0.01 wt % to about 10 wt % of a glyceryl ester of a fatty acid,

wherein the ester of a C₂₋₄ alcohol and a fatty acid is not isopropyl myristate.

Some embodiments provide for a topical composition comprising:

(a) about 0.01 wt % to about 2.5 wt % of a corticosteroid;

(b) an ester of a C₂₋₄ alcohol and a fatty acid;

(c) about 10 wt % to about 80 wt % of a C₂₋₄ alcohol;

(d) about 3 wt % to about 75 wt % of an oleaginous vehicle selected from mineral oil, soybean oil, caprylic/capric triglycerides, and a combination thereof; and

(e) about 0.01 wt % to about 10 wt % of a glyceryl ester of a fatty acid, wherein the ester of a C₂₋₄ alcohol and a fatty acid is less than about 9 wt % isopropyl myristate.

Some embodiments provide for a composition comprising:

(a) about 0.01 wt % to about 2.5 wt % desoximetasone;

(b) isopropyl palmitate;

(c) about 10 wt % to about 80 wt % C₂₋₄ alcohol;

(d) about 3 wt % to about 75 wt % of an oleaginous vehicle selected from mineral oil, soybean oil, caprylic/capric triglycerides, and a combination thereof; and

(e) about 0.01 wt % to about 10 wt % of glyceryl monooleate.

Some embodiments provide for a composition comprising:

(a) about 0.01 wt % to about 2.5 wt % of a corticosteroid;

(b) about 10% to 80 wt % C₂₋₄ alcohol;

(c) about 0.01 wt % to about 10 wt % of glyceryl monooleate;

(d) about 3 wt % to about 80 wt % of an oleaginous vehicle selected from mineral oil, soybean oil, caprylic/capric triglycerides, and a combination thereof; and

(e) an ester of a C₂₋₄ alcohol and a fatty acid; wherein the ester of a C₂₋₄ alcohol and a fatty acid is not isopropyl myristate.

### BRIEF DESCRIPTION OF THE DRAWINGS

FIG. 1 summarizes human skin permeation and retention results of exemplary formulations, TrDsx37 and TrDsx39, versus comparative example, Control. The delivered dose of desoximetasone (μg/cm²) is shown at 3 hours, 6 hours, and 24 hours and as measured after 24 hours in the epidermis and dermis.

FIG. 2 summarizes human skin permeation and retention results of exemplary formulations, TrDsx37, TrDsx62, and TrDsx63, versus comparative example, Control. The delivered dose of desoximetasone (μg/cm) is shown at 4 hours and 24 hours and as measured after 24 hours in the epidermis and dermis.

FIG. 3 summarizes human skin permeation and retention results of exemplary formulations, TrDsx37, TrDsx62, and TrDsx64, versus comparative example, Control. The delivered dose of desoximetasone (1 g/cm²) is shown at 4 hours and 24 hours and as measured after 24 hours in the epidermis and dermis.

FIG. 4 summarizes human skin permeation and retention results of exemplary formulations, TrDsx37, TrDsx70, TrDsx71, TrDsx72, and TrDsx73, versus comparative example, Control. The delivered dose of desoximetasone (μ/cm²) is shown at 4 hours and 24 hours and as measured after 24 hours in the epidermis and dermis.

FIG. 5 summarizes human skin permeation and retention results of exemplary formulations, TrDsx73, TrDsx82, TrDsx83, and TrDsx84, versus comparative example, Control. The delivered dose of desoximetasone (μg/cm²) is shown at 3 hours, 6 hours, and 24 hours and as measured after 24 hours in the epidermis and dermis.

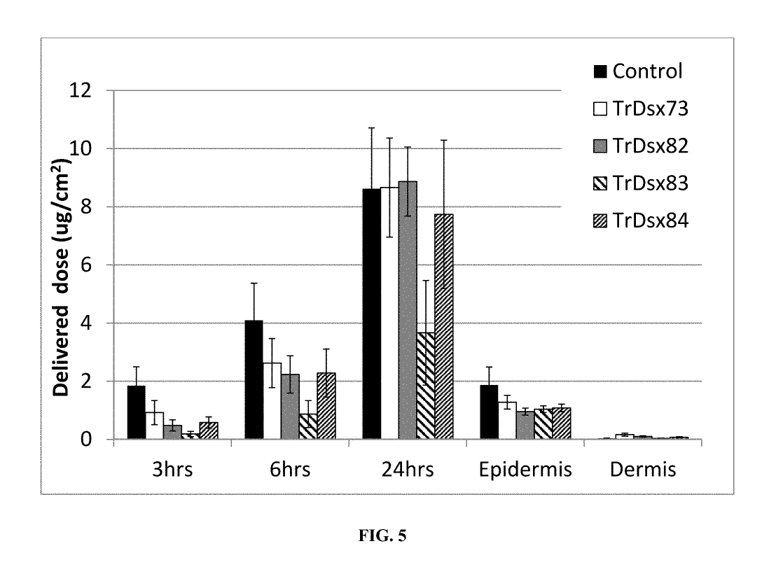

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

Further, the singular forms “a,” “an,” and “the” include plural references unless the context clearly dictates otherwise. Thus, reference to “the compound” includes a plurality of such compounds, and reference to “the assay” includes reference to one or more assays and equivalents thereof known to those skilled in the art.

Recitation of numeric ranges of values throughout the disclosure is intended to serve as a shorthand notation of referring individually to each separate value falling within the range inclusive of the values defining the range, and each separate value is incorporated in the specification as it were individually recited herein.

The term “wt %” or, equivalently, “% w/w” as used herein refers to the weight of a component based on the total weight of a composition comprising the component. For example, if component A is present in an amount of 50% w/w in a 100 mg composition, component A is present in an amount of 50 mg.

The term “about” as used herein to modify a numerical value indicates a defined range around that value. If “X” were the value, “about X” would generally indicate a value from 0.95X to 1.05X. When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11%” is equivalent to “about 7%, about 9%, or about 11%.”

### Compositions

Provided herein are compositions comprising an active agent for topical administration that are capable of administering an effective amount of the active agent through the barrier function of the skin. In some embodiments, the active agent is a corticosteroid useful for the treatment of a skin disease or condition.

In some embodiments, the active agent is a natural corticosteroid, a progesterone-type synthetic corticosteroid, a hydrocortisone-type synthetic corticosteroid, a methasone-type synthetic corticosteroid, a synthetic corticosteroid that is an acetonide or a related compound, or another synthetic corticosteroid.

Suitable corticosteroids include a natural corticosteroid such as: 11-dehydrocorticosterone, 11-deoxycorticosterone, 11-deoxycortisol, 11-ketoprogesterone, 11-hydroxypregnenolone, 11-hydroxyprogesterone, 11β,17α,21-trihydroxypregnenolone, 17α,21-dihydroxypregnenolone, 17α-hydroxypregnenolone, 17α-hydroxyprogesterone, 18-hydroxy-1-deoxycorticosterone, 18-hydroxycorticosterone, 18-hydroxyprogesterone, 21-deoxycortisol, 21-deoxycortisone, 21-hydroxypregnenolone, aldosterone, corticosterone, cortisol, cortisone, pregnenolone, progesterone; a synthetic corticosteroid of progesterone-type such as: flugestone, fluorometholone, medrysone, prebediolone acetate; a synthetic corticosteroid of hydrocortisone-type such as: chloroprednisone, cloprednol, difluprednate, fludrocortisone, fluocinolone, fluperolone, fluprednisolone, loteprednol, m ethylprednisolone, prednicarbate, prednisolone, prednisone, tixocortol, triamcinolone; a synthetic corticosteroid of methasone (16-methylated) type such as: dexamethasone, alclometasone, beclometasone, betamethasone, clobetasol, clobetasone, clocortolone, desoximetasone, dexamethasone, diflorasone, difluocortolone, fluclorolone, flumetasone, fluocortin, fluocortolone, fluprednidene, fluticasone, fluticasone furoate, halometasone, meprednisone, mometasone, mometasone furoate, paramethasone, prednylidene, rimexolone, ulobetasol; a synthetic corticosteroid that is an acetonide or a related compound such as: amcinonide, budesonide, ciclesonide, deflazacort, desonide, fornmocortal, fluclorolone acetonide, fludroxycortide, flunisolide, fluocinolone acetonide, fluocinonide, halcinonide, triamcinolone acetonide; or another synthetic corticosteroid such as cortivazol or RU-28362.

In some embodiments, the active agent is an agent used for the treatment of psoriasis, including but not limited to halobetasol, clobetasol propionate, and mometasone furoate which have also been formulated in spray formats for the treatment of psoriasis in several patents. In some embodiments, the corticosteroid is betamethasone dipropionate. In some embodiments, the corticosteroid is desoximetasone.

Some embodiments herein provide for compositions, comprising an active agent for topical administration, that are stable for at least 1 week, 2 weeks, 3 weeks, or 4 weeks. The term “stable” refers to physical stability and/or chemical stability of the active agent tinder certain temperatures or humidity levels as described herein.

In some embodiments, the composition is stable for at least 2 weeks at room temperature. In some embodiments, the composition is stable for at least 3 weeks at room temperature. In some embodiments, the composition is stable for at least 4 weeks at room temperature. In some embodiments, the composition is stable for at least 2 weeks at 40° C. In some embodiments, the composition is stable for at least 3 weeks at 40° C. In some embodiments, the composition is stable for at least 4 weeks at 40° C.

In some embodiments, the composition is stable for at least 2 weeks under increased humidity (including but not limited to, greater than about 50%, 60%, 70%, or 75% humidity). In some embodiments, the composition is stable for at least 3 weeks under increased humidity. In some embodiments, the composition is stable for at least 4 weeks under increased humidity.

In some embodiments, the active agent degrades by less than about 1% over the course of 1 month, 2 months, 3 months, 4 months, 5 months, or 6 months at room temperature. In some embodiments, the active agent degrades by less than about 0.9, 0.8, 0.7, 0.6, 0.5, 0.4, 0.3, 0.2, or 0.1% over the course of 1 month, 2 months, 3 months, 4 months, 5 months, or 6 months at room temperature.

Some embodiments provide for a topical composition comprising an active agent and an ester of a C₂₋₄ alcohol and a fatty acid, wherein an effective amount of the active agent is delivered to the dermis or epidermis for the treatment of a skin disease.

Some embodiments provide for a topical composition comprising an active agent and an ester of a C₂₋₄ alcohol and a fatty acid, wherein the active agent is delivered to the dermis at a dose of greater than about 1 μg of active agent per cm² area of skin in up to 24 hours. Some embodiments provide for a topical formulation comprising desoximetasone and an ester of a C₂₋₄ alcohol and a fatty acid, wherein desoximetasone is delivered to the dermis at a dose of greater than about 1 μg of desoximetasone per cm² area of skin in up to 24 hours.

In some embodiments, the active agent is delivered to the dermis at a dose of greater than about 0.5 μg of active agent per cm² area of skin. In some embodiments, the active agent is delivered to the dermis at a dose of greater than about 0.5 μg of active agent per cm² area of skin in at least 24 hours. In some embodiments, the active agent is delivered to the dermis at a dose from about 0.2 μg of active agent per cm² area of skin to about 2.5 μg of active agent per cm² area of skin. In some embodiments, the active agent is delivered to the dermis at a dose from about 0.2 μg of active agent per cm² area of skin to about 2.5 μg of active agent per cm² area of skin in at least about 24 hours. In some embodiments, the active agent is delivered to the dermis at a dose from about 1 μg of active agent per cm² area of skin to about 2 μg of active agent per cm² area of skin. In some embodiments, the active agent is delivered to the dermis at a dose from about 1 μg of active agent per cm² area of skin to about 2 μg of active agent per cm² area of skin in at least about 24 hours.

In some embodiments, the active agent is delivered to the epidermis at a dose of greater than about 0.2 μg of active agent per cm² area of skin. In some embodiments, the active agent is delivered to the epidermis at a dose of greater than about 0.2 μg of active agent per cm² area of skin in at least 24 hours. In some embodiments, the active agent is delivered to the epidermis at a dose of from about 0.25 μg of active agent per cm² area of skin to about 2.5 μg of active agent per cm² area of skin. In some embodiments, the active agent is delivered to the epidermis at a dose of from about 0.25 μg of active agent per cm² area of skin to about 2.5 μg of active agent per cm² area of skin in at least 24 hours. In some embodiments, the active agent is delivered to the epidermis at a dose of from about 0.5 μg of active agent per cm² area of skin to about 2 μg of active agent per cm² area of skin in at least 24 hours. In some embodiments, the active agent is delivered to the epidermis at a dose of from about 0.5 μg of active agent per cm² area of skin to about 2 μg of active agent per cm² area of skin in at least 24 hours.

In some embodiments, the active agent is desoximetasone.

In some embodiments, the ester of a C₂₋₄ alcohol and a fatty acid is present in an amount of about 2 wt % to about 12 wt %. In some embodiments, the ester of a C₂₋₄ alcohol and a fatty acid is isopropyl palmitate. In some embodiments, isopropyl palmitate is present in an amount of about 2 wt % to about 10 wt %. In some embodiments, isopropyl palmitate is present in an amount of less than about 10 wt %. In some embodiments, the ester of a C₂₋₄ alcohol and a fatty acid is not isopropyl myristate.

In some embodiments, the composition comprises an oleaginous vehicle. The term “oleaginous” is defined as “having the nature or qualities of oil.” The term “oleaginous vehicle” as used herein refers to a composition that has the organoleptic character of an oily substance, i.e., oily feeling, when topically administered to the skin. Suitable oleaginous vehicles include Alkyl (C₁₂-C₁₅) benzoate NF, almond oil NF, coconut oil, corn oil NF, cottonseed oil, ethyl oleate, isopropyl myristate, isopropyl palmitate, mineral oil, light mineral oil, octyldodecanol, olive oil, peanut oil, safflower oil, sesame oil, soybean oil, squalene, caprylic/capric triglycerides (such as CRODAMOL™ GTCC) and combinations thereof. Mineral oil and light mineral oil are herein both referred to as mineral oil.

In some embodiments, the oleaginous vehicle is selected from mineral oil, soybean oil, caprylic/capric triglycerides, and a combination thereof.

In some embodiments, the composition comprises a penetration enhancer. A “penetration enhancer” or “molecular penetration enhancer” or “MPE™” as used herein includes an agent or a combination of agents that improves the transport of molecules such as a pharmaceutically or cosmetically active agent into or through a natural membrane such as skin or nail. In this specification the terms “penetration enhancer,” “chemical penetration enhancer,” “molecular penetration enhancer” and “MPE™” are used interchangeably.

Penetration enhancers include, but are not limited to, alpha-terpineol, alpha-tocopherol, ammonium lauryl sulfate, Choleth-24, cocodiethanolamide, coco-caprylate/caprate, dichlorodifluororethane, diethanolamine, diethylsebacate, diethylene glycol monomethyl ether, diisopropanolamine, diisopropyl adipate, diisopropyl dilinoleate, dimethyl isosorbide, dimethyl sulfoxide (“DMSO”), dipropylene glycol, ethyl acetate, ethyl oleate, ethylene glycol, fatty acids, glycerin, glyceryl isostearate, glyceryl laurate, glyceryl monostearate, glyceryl monoleate (Capmul® GMO-50), glyceryl palmitate, glyceryl rincoleate, glyceryl stearate-laureth 23, hexylene glycol, hydrogenated castor oil, imidurea, isoceteth-20, isopropyl alcohol, isopropyl isostearate, isopropyl myristate, isopropyl palmitate, lactic acid, lauramnine oxide, laureth-2, laureth-23, laureth-4, lauric diethanolomide, lauric/myristic diethanololarnide, lauryl acetate, L-menthol, levulinic acid, (+/−)-limonene, methoxy PEG-16, methyl alcohol, methyl gluceth-10, methyl laurate, methyl salicylate, myristyl alcohol, myristyl lactate, octyldodecanol, oleic acid, oleth-10, oleth-5, oleth-2, oleth-20, oleyl alcohol, oleyl oleate, PEG-60 hydrogenated castor oil, PEGmethyl ether, pentadecalactone, polyoxyl 40 hydrogenated castor oil, polysorbate 20, polysorbate 40, polysorbate 60, polysorbate 65, polysorbate 80, propylene carbonate, propylene glycol, propylene glycol diacetate, propylene glycol dicaprylate, propylene glycol monolaurate, propylene glycol monopalmitostearate, SD alcohol 408, sodium lactate, sodium laureth-2 sulfate, sodium laureth-3 sulfate, sodium lauryl sulfate, sorbitan isostearate, sorbitan monolaurate, sorbitan monooleate, sorbitan monoplamitate, sorbitan monostearate, sorbitan sesquioleate, sorbitan tristearate, spermaceti, squalene, steareth-10, steareth-100, steareth-2, steareth-20, steareth-21, steareth-40, tocopherol, trideceth-110, triethanolamine lauryl sulfate, trolamine, urea, polyethylene glycol 400, cyclomethicone, ceteareth-12, ceteareth-15, ceteareth-30, ceteth-10, ceteth-2, ceteth-20, ceteth-23, and medium chain triglycerides.

In some embodiments, the penetration enhancer is isopropyl myristate, isopropyl palmitate, transcutol, oleic acid, propylene glycol, 1,3-butanediol, butylene dioxide, hexylene glycol, dimethyl sulfoxide, soybean oil, corn oil, caprylic/capric triglycerides, glycerol, lauryl lactate, Labrasol®, castor oil, sorbitol, or a combination thereof.

In some embodiments, the composition further comprises about 10 wt % to about 80 wt % C₂₋₄ alcohol. In some embodiments, the C₂₋₄ alcohol is ethanol. In some embodiments, the C₂₋₄ alcohol is isopropyl alcohol.

In some embodiments, the composition does not include polyethylene glycol.

Some embodiments provide for a topical composition comprising or consisting essentially of:

(a) about 0.01 wt % to about 2.5 wt % of a corticosteroid;

(b) an ester of a C₂₋₄ alcohol and a fatty acid;

(c) about 10 wt % to about 80 wt % C₂₋₄ alcohol;

(d) about 3 wt % to about 75 wt % of an oleaginous vehicle; and

(e) about 0.01 wt % to about 10 wt % of a glyceryl ester of a fatty acid,

wherein the ester of a C₂₋₄ alcohol and a fatty acid is not isopropyl myristate.

Some embodiments provide for a topical composition comprising or consisting essentially of:

(a) about 0.01 wt % to about 2.5 wxt % of a corticosteroid;

(b) an ester of a C₂₋₄ alcohol and a fatty acid;

(c) about 10 wt % to about 80 wt % C₂₋₄ alcohol;

(d) about 3 wt % to about 75 wt % of an oleaginous vehicle; and

(e) about 0.01 wt % to about 10 wt % of a glyceryl ester of a fatty acid,

wherein the ester of a C₂₋₄ alcohol and a fatty acid is less than about 9 wt % isopropyl myristate.

Some embodiments provide for a topical composition comprising or consisting essentially of:

(a) about 0.01 wt % to about 2.5 wt % of a corticosteroid;

(b) an ester of a C₂₋₄ alcohol and a fatty acid;

(c) about 10 wt % to about 80 wt % C₂₋₄ alcohol;

(d) about 3 wt % to about 75 wt % of an oleaginous vehicle selected from mineral oil, soybean oil, caprylic/capric triglycerides, and a combination thereof; and

(e) about 0.01 wt % to about 10 wt % of a glyceryl ester of a fatty acid, wherein the ester of a C₂₋₄ alcohol and a fatty acid is not isopropyl myristate.

Some embodiments provide for a topical composition comprising or consisting essentially of:

(a) about 0.01 wt % to about 2.5 wt % of a corticosteroid;

(b) an ester of a C₂₋₄ alcohol and a fatty acid;

(c) about 10 wt % to about 80 wt % C₂₋₄ alcohol;

(d) about 3 wt % to about 75 wt % of an oleaginous vehicle selected from mineral oil, soybean oil, caprylic/capric triglycerides, and a combination thereof; and

(e) about 0.01 wt % to about 10 wt % of a glyceryl ester of a fatty acid,

wherein the ester of a C₂₋₄ alcohol and a fatty acid is less than about 9 wt % isopropyl myristate.

In some embodiments, the corticosteroid is desoximetasone. In some embodiments, the desoximetasone is present in about 0.01 wt % to about 2.5 wt %.

In some embodiments, the ester of a C₂₋₄ alcohol and a fatty acid is present in an amount of about 2 wt % to about 12 wt %. In some embodiments, the ester of a C₂₋₄ alcohol and a fatty acid is present in an amount of less than 10 wt %.

In some embodiments, the ester of a C₂₋₄ alcohol and a fatty acid is isopropyl palmitate.

In some embodiments, the C₂₋₄ alcohol is isopropyl alcohol. In some embodiments, the isopropyl alcohol is present in an amount of about 15 wt % to about 50 wt %.

In some embodiments, the glyceryl ester of a fatty acid is glyceryl monostearate, glyceryl isostearate, glyceryl palmitate, or glyceryl ricinoleate. In some embodiments, the glyceryl ester of a fatty acid is glyceryl monooleate.

In some embodiments, the oleaginous vehicle is mineral oil. In some embodiments, the oleaginous vehicle is soybean oil. In some embodiments, the oleaginous vehicle is a combination of mineral oil and soybean oil. In some embodiments, the oleaginous vehicle is a combination of mineral oil, soybean oil, and caprylic/capric triglycerides.

In some embodiments, the topical composition further comprises of 0.001 wt % to about 5% menthol. In some embodiments, the menthol is present in an amount of about 0.02 wt % to about 0.3 wt %. In some embodiments, the menthol is present in an amount of about 0.01 wt % to about 1 wt %.

Some embodiments provide for a composition comprising:

(a) about 0.01 wt % to about 2.5 wt % desoximetasone;

(b) isopropyl palmitate;

(c) about 10 wt % to about 80 wt % C₂₋₄ alcohol;

(d) about 3 wt % to about 75 wt % of an oleaginous vehicle selected from mineral oil, soybean oil, caprylic/capric triglycerides, and a combination thereof; and

(e) about 0.01 wt % to about 10 wt % of glyceryl monooleate.

In some embodiments, desoximetasone is present in an amount of about 0.01 wt % to about 2.5 wt %. In some embodiments, desoximetasone is present in an amount of about 0.1 wt % to about 1.5 wt %. In some embodiments, desoximetasone is present in an amount of about 0.15 wt % to about 1 wt %. In some embodiments, desoximetasone is present in an amount of about 0.2 wt % to about 0.5 wt %. In some embodiments, desoximetasone is present in an amount of about 0.2 wt % to about 0.3 wt %.

In some embodiments, the desoximetasone is present in an amount of about 0.5 wt %. In some embodiments, the desoximetasone is present in an amount of about 0.45 wt %. In some embodiments, the desoximetasone is present in an amount of about 0.4 wt %. In some embodiments, the desoximetasone is present in an amount of about 0.35 wt %. In some embodiments, the desoximetasone is present in an amount of about 0.3 wt %. In some embodiments, the desoximetasone is present in an amount of about 0.25 wt %. In some embodiments, the desoximetasone is present in an amount of about 0.2 wt %. In some embodiments, the desoximetasone is present in an amount of about 0.15 wt %. In some embodiments, the desoximetasone is present in an amount of about 0.1 wt %. In some embodiments, the desoximetasone is present in an amount of about 0.05 wt %.

In some embodiments, isopropyl palmitate is present in an amount of about 0.1 wt % to about 75 wt %. In some embodiments, isopropyl palmitate is present in an amount of about 1 wt % to about 70 wt %, In some embodiments, isopropyl palmitate is present in an amount of about 2 wt % to about 50 wt %.

In some embodiments, isopropyl palmitate is present in an amount of about 2 wt % to about 15 wt %. In some embodiments, isopropyl palmitate is present in an amount of about 2 wt % to about 12 wt %. In some embodiments, isopropyl palmitate is present in an amount of about 5 wt % to about 10 wt %. In some embodiments, isopropyl palmitate is present in an amount of about 3 wt % to about 8 wt %. In some embodiments, isopropyl palmitate is present in an amount of about 2 wt % to about 10 wt %. In some embodiments, isopropyl palmitate is present in an amount of about 5 wt % to about 12 wt %.

In some embodiments, isopropyl palmitate is present in an amount of greater than 1 wt %. In some embodiments, isopropyl palmitate is present in an amount of greater than 2 wt %. In some embodiments, isopropyl palmitate is present in an amount of greater than 3 wt %. In some embodiments, isopropyl palmitate is present in an amount of greater than 4 wt %. In some embodiments, isopropyl palmitate is present in an amount of greater than 5 wt %.

In some embodiments, isopropyl palmitate is present in an amount of less than 70 wt %. In some embodiments, isopropyl palmitate is present in an amount of less than 50 wt %. In some embodiments, isopropyl palmitate is present in an amount of less than 40 wt %. In some embodiments, isopropyl palmitate is present in an amount of less than 30 wt %. In some embodiments, isopropyl palmitate is present in an amount of less than 25 wt %. In some embodiments, isopropyl palmitate is present in an amount of less than 20 wt %. In some embodiments, isopropyl palmitate is present in an amount of less than 15 wt %. In some embodiments, isopropyl palmitate is present in an amount of less than 10 wt %.

Some embodiments of the disclosure provide for compositions comprising a C₂₋₄ alcohol. In some embodiments, the C₂₋₄ alcohol is ethanol. In some embodiments, the C₂₋₄ alcohol is n-propyl alcohol. In some embodiments, the C₂₋₄ alcohol is isopropyl alcohol.

In some embodiments, the C₂₋₄ alcohol is present in an amount of about 10 wt % to about 80 wt %. In some embodiments, the C₂₋₄ alcohol is present in an amount of about 15 wt % to about 75 wt %. In some embodiments, the C₂₋₄ alcohol is present in an amount of about 20 wt % to about 70 wt %. In some embodiments, the C₂₋₄ alcohol is present in an amount of about 15 wt % to about 50 wt %. In some embodiments, the C₂₋₄ alcohol is present in an amount of about 20 wt % to about 30 w t %.

In some embodiments, wherein the C₂₋₄ alcohol is isopropyl alcohol, the isopropyl alcohol is present in an amount of about 10 wt % to about 80 wt %. In some embodiments, isopropyl alcohol is present in an amount of about 15 wt % to about 50 wt %. In some embodiments, the isopropyl alcohol is present in an amount of about 20 wt % to about 30 wt %.

In some embodiments, the glyceryl monooleate is present in an amount of about 0.01 wt % to about 10 wt %. In some embodiments, the glyceryl monooleate is present in an amount of about 0.01 wt % to about 5 wt %. In some embodiments, the glyceryl monooleate is present in an amount of about 0.1 wt % to about 9 wt %. In some embodiments, the glyceryl monooleate is present in an amount of about 0.25 wt % to about 8 wt %. In some embodiments, the glyceryl monooleate is present in an amount of about 0.5 wt % to about 7 wt %. In some embodiments, the glyceryl monooleate is present in an amount of about 0.75 wt % to about 8 wt %. In some embodiments, the glyceryl monooleate is present in an amount of about 1 wt % to about 5 wt %.

In some embodiments, the oleaginous vehicle is a mineral oil, soybean oil, caprylic/capric triglycerides, or a combination thereof. In some embodiments, the oleaginous vehicle is a mineral oil. In some embodiments, the oleaginous vehicle is soybean oil. In some embodiments, the oleaginous vehicle is a combination of mineral oil and soybean oil. In some embodiments, the oleaginous vehicle is a combination of mineral oil, soybean oil, and caprylic/capric triglycerides.

In some embodiments, the compositions comprise about 3 wt % to about 80 wt % of an oleaginous vehicle. In some embodiments, the compositions comprise about 3 wt % to about 75 wt % of an oleaginous vehicle. In some embodiments, the oleaginous vehicle is present in an amount of about 10 wt % to about 75 wt %. In some embodiments, the oleaginous vehicle is present in an amount of about 20 wt % to about 75 wt %. In some embodiments, the oleaginous vehicle is present in an amount of about 30 wt % to about 75 wt %. In some embodiments, the oleaginous vehicle is present in an amount of about 40 wt % to about 75 wt %. In some embodiments, the oleaginous vehicle is present in an amount of about 50 wt % to about 70 wt %. In some embodiments, the oleaginous vehicle is present in an amount of about 65 wt % to about 75 wt %. In some embodiments, the oleaginous vehicle is present in an amount of about 70 wt % to about 80 wt %. In some embodiments, the oleaginous vehicle is present in an amount of about 30 wt % to about 80 wt %.

In some embodiments, the compositions described herein further comprise menthol. In some embodiments, the compositions comprise 0.001 wt % to about 5 wt % menthol. In some embodiments, the menthol is present in an amount of about 0.01 wt % to about 2.5 wt %. In some embodiments, the menthol is present in an amount of about 0.01 wt % to about 1 wt %. In some embodiments, the menthol is present in an amount of about 0.015 wt % to about 0.5 wt %. In some embodiments, the menthol is present in an amount of about 0.02 wt % to about 0.3 wt %.

In some embodiments, the compositions comprise about 0.1 wt % to about 5 wt % of lauryl lactate. In some embodiments, the compositions comprise about 0.5 wt % to about 3 wt % of lauryl lactate.

In some embodiments, the compositions described herein comprise less than about 40 wt % of isopropyl myristate. In some embodiments, the compositions described herein comprise less than about 10 wt % of isopropyl myristate. In some embodiments, the compositions described herein comprise less than about 9 wt % of isopropyl myristate. In some embodiments, the compositions described herein comprise less than about 5 wt % of isopropyl myristate. In some embodiments, the compositions described herein comprise less than about 1 wt % of isopropyl myristate. In some embodiments, the compositions described herein do not include isopropyl myristate.

Some embodiments provide for a composition comprising:

(a) about 0.01 wt % to about 2.5 wt % of a corticosteroid;

(b) about 10% to 80 wt % C₂₋₄ alcohol:

(c) about 0.01 wt % to about 10 wt % of glyceryl monooleate;

(d) about 3 wt % to about 80 wt % of an oleaginous vehicle selected from mineral oil, soybean oil, caprylic/capric triglycerides, and a combination thereof; and

(e) an ester of a C₂₋₄ alcohol and a fatty acid; wherein the ester of a C₂₋₄ alcohol and a fatty acid is not isopropyl myristate.

Some embodiments provide for a composition comprising:

(a) about 0.01 wt % to about 2.5 wt % of a corticosteroid;

(b) about 10% to 80 wt % C₂₋₄ alcohol;

(c) about 0.01 wt % to about 10 wt % of glyceryl monooleate;

(d) about 3 wt % to about 80 wt % of an oleaginous vehicle selected from mineral oil, soybean oil, caprylic/capric triglycerides, and a combination thereof, and

(e) an ester of a C₂₁ alcohol and a fatty acid; wherein the ester of a C₂₋₄ alcohol and a fatty acid is less than about 9 wt % isopropyl myristate.

In some embodiments, the compositions further comprise caprylic/capric triglycerides.

In some embodiments, the caprylic/capric triglycerides are present in an amount of about 0.5 wt % to about 50 wt %. In some embodiments, the caprylic/capric triglycerides are present in an amount of about 1 wt % to about 40 wt %. In some embodiments, the caprylic/capric triglycerides are present in an amount of about 2.5 wt % to about 30 wt %. In some embodiments, the caprylicicapric triglycerides are present in an amount of about 2.5 wt % to about 10 wt %.

Some embodiments herein provide for a composition as described in Tables 1 to 7.

In some embodiments, provided is a topical formulation comprising desoximetasone that provides a similar profile of delivery of desoximetasone to dermal tissue of a subject as a target formulation comprising about 0.25 wt % desoximetasone, glyceryl oleate, about 23 wt % isopropyl alcohol, about 32 wt % isopropyl myristate, less than about 1 wt % L-menthol, and mineral oil; and wherein the topical formulation comprises less than 9% w/w of isopropyl myristate. In some embodiments, provided is a topical formulation comprising desoximetasone that provides a similar profile of delivery of desoximetasone to dermal tissue of a subject as a target formulation comprising about 0.25 wt % desoximetasone, glyceryl oleate, about 23 wt % isopropyl alcohol, about 32 wt % isopropyl myristate, less than about 1 wt % L-menthol, and mineral oil; and wherein the topical formulation comprises no isopropyl myristate. In some embodiments, the mineral oil is present in an amount of about 44 wt %. In some embodiments, the glyceryl oleate is present in an amount of about 0.9 wt %.

In some embodiments, provided is a topical formulation comprising desoximetasone that provides a similar profile of delivery of desoximetasone to dermal tissue of a subject as a target formulation comprising about 0.25 wt % desoximetasone, glyceryl monoleate, about 23 wt % isopropyl alcohol, about 32 wt % isopropyl myristate, less than about 1 wt % L-menthol, and mineral oil; and wherein the topical formulation comprises less than 9% w/w of isopropyl myristate. In some embodiments, provided is a topical formulation comprising desoximetasone that provides a similar profile of delivery of desoximetasone to dermal tissue of a subject as a target formulation comprising about 0.25 wt % desoximetasone, glyceryl monoleate, about 23 wt % isopropyl alcohol, about 32 wt % isopropyl myristate. less than about 1 wt % L-menthol, and mineral oil; and wherein the topical formulation comprises no isopropyl myristate.

In some embodiments, provided is a topical formulation comprising desoximetasone that provides a similar profile of delivery of desoximetasone to epidermal tissue or through the skin of a subject as a target formulation comprising about 0.25 wt % desoximetasone, glyceryl oleate, about 23 wt % isopropyl alcohol, about 32 wt % isopropyl myristate, less than about 1 wt % L-menthol, and mineral oil; and wherein the topical formulation comprises less than 9% w/w of isopropyl myristate. In some embodiments, provided is a topical formulation comprising desoximetasone that provides a similar profile of delivery of desoximetasone to epidermal tissue or through the skin of a subject as a target formulation comprising about 0.25 wt % desoximetasone, glyceryl oleate, about 23 wt % isopropyl alcohol, about 32 wt % isopropyl myristate, less than about 1 wt % L-menthol, and mineral oil; and wherein the topical formulation comprises no isopropyl myristate. In some embodiments, the mineral oil is present in an amount of about 44 wt %. In some embodiments, the glyceryl oleate is present in an amount of about 0.9 wt %.

In some embodiments, provided is a topical formulation comprising desoximetasone that provides a similar profile of delivery of desoximetasone to epidermal tissue or through the skin of a subject as a target formulation compri sing about 0.25 wt % desoximetasone, glyceryl monoleate, about 23 wt % isopropyl alcohol, about 32 wt % isopropyl myristate, less than about 1 wt % L-menthol, and mineral oil; and wherein the topical formulation comprises less than 9% w/w of isopropyl myristate. In some embodiments, provided is a topical formulation comprising desoximetasone that provides a similar profile of delivery of desoximetasone to epidermal tissue or through the skin of a subject as a target formulation comprising about 0.25 wt % desoximetasone, glyceryl monoleate, about 23 wt % isopropyl alcohol, about 32 wt % isopropyl myristate, less than about 1 wt % L-menthol, and mineral oil; and wherein the topical formulation comprises no isopropyl myristate.

“A similar profile of delivery of desoximetasone” indicates that a formulation delivers an amount of desoximetasone about equal to the amount of desoximetasone delivered by the target formulation. In some embodiments, a formulation delivers an amount of desoximetasone about equal to the amount of desoximetasone delivered by the target formulation in about an equal amount of time. In some embodiments, the topical formulation that provides a similar profile of delivery of desoximetasone as a target formulation comprises a topical formulation as disclosed herein.

Also provided herein are methods for preparing compositions described herein. In some embodiments, the methods comprise dissolving the active agent in the C₂₋₄ alcohol and adding the remaining components. In some embodiments, a method of making a composition described herein comprises: (a) dissolving the desoximetasone in the C₂₋₄ alcohol; and (b) adding the remaining components (i.e. isopropyl palmitate, the oleaginous vehicle, and the glyceryl monooleate). In some embodiments, a method for preparing a composition described herein comprises: (a) dissolving the desoximetasone in the C₂₋₄ alcohol; and (b) adding the isopropyl palmitate, the oleaginous vehicle, and the glyceryl monooleate.

Some embodiments herein provide for a method of identifying a topical composition useful for the treatment of skin disease comprising:

(a) measuring a property of an active agent of a target composition;

(b) preparing a test composition comprising the active agent and one or more components of the target composition;

(c) measuring the property of the active agent of the test composition;

(d) comparing the property of the active agent of the test composition with the property of the active agent of the target composition; and

(e) modifying one or more components of the test composition.

In some embodiments, the target composition is a composition known to be useful for treating skin disease. In some embodiments, modifying one or more components of the test composition comprises adding a component, removing a component, adding or removing an amount of a component, or a combination thereof.

In some embodiments, the property to be measured and compared is delivery of the active agent to the dermis of a subject, delivery of the active agent to the epidermis of a subject, stability of the active agent, delivery of the active agent through the epidermis and dermis of a subject, amount of active agent delivered via spraying (such as through a spray head using a manually-actuated or powered pump or a propellant gas in a pressurized canister), or a combination thereof.

In some embodiments, the method of identifying a topical composition further comprises repeating steps (a) to (e) until the property of the active agent of the test composition is about equal to, or greater than, the property of the active agent of the target composition.

In some embodiments, the test composition comprises desoximetasone. In some embodiments, the target composition does not include isopropyl myristate.

Some embodiments herein provide for a method of identifying a topical formulation as disclosed herein, comprising:

(a) measuring stability of desoximetasone of a target formulation;

(b) preparing a test composition comprising desoximetasone and one or more excipients of the target formulation;

(c) measuring stability of desoximetasone of the test composition;

(d) comparing the stability of desoximetasone of the test composition with the delivery of desoximetasone of the target formulation; and

(e) modifying one or more excipients of the test composition.

In some embodiments, steps (a) to (e) above are repeated until the topical formulation as disclosed herein is achieved. In some embodiments, steps (a) to (e) above are repeated until the stability of desoximetasone of the test composition is equal to, or greater than, the stability of desoximetasone of the target formulation. In some embodiments, stability of the target formulation or stability of the test composition is measured as amount of active agent degraded over time. In some embodiments, stability of the target formulation or stability of the test composition is measured at room temperature, about 30° C., or about 40° C. over time (i.e., 1 week, 2 weeks, 3 weeks, 4 weeks, etc). In some embodiments, stability of the target formulation or stability of the test composition is measured at increased humidity (greater than about 50%, 60%, 70%, or 75% humidity) over time (i.e., 1 week, 2 weeks, 3 weeks, 4 weeks, etc).

In some embodiments, the target formulation does not include ispropyl myristate.

Some embodiments herein provide for a method of identifying a topical formulation as disclosed herein, comprising:

(a) measuring delivery of desoximetasone of the target formulation;

(b) preparing a test composition comprising desoximetasone and one or more excipients of the target formulation;

(c) measuring delivery of desoximetasone of the test composition;

(d) comparing the delivery of desoximetasone of the test composition with the delivery of desoximetasone of the target formulation; and

(e) modifying one or more excipients of the test composition.

In some embodiments, steps (a) to (e) above are repeated until the topical formulation as disclosed herein is achieved. In some embodiments, steps (a) to (e) above are repeated until the delivery of desoximetasone of the test composition is equal to, or greater than, the delivery of desoximetasone of the target formulation. In some embodiments, the delivery of desoximetasone is measured as delivery of the desoximetasone to the dermis of a subject. In some embodiments, the delivery of desoximetasone is measured as delivery of the desoximetasone to the epidermis of a subject. In some embodiments, the delivery of desoximetasone is measured as amount of desoximetasone dispensed by a spray.

In some embodiments, the target formulation does not include ispropyl myristate.

### Formulations

The compositions provided herein are usually administered in the form of formulations. Thus, provided herein are also pharmaceutical formulations that contain one or more of the compositions described herein and may further comprise one or more pharmaceutically acceptable carriers, adjuvants and excipients.

Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and

Methods of preparing compositions for topical administration are known in the art (see, for example, Remington: The Science and Practice of Pharmacy, 21st Edition Edited by the Philadelphia College of Pharmacy and Science editorial board. Published by Lippincott Williams and Wilkins, Baltimore, Md., 2006. ISBN 0-7817-4675-6; The United States Pharmacopeia: The National Formulary, USP37 NF32, 2014. ISBN 10: 936424223; and Modern Pharmaceutics, Two Volume Set, Fifth Edition (Drugs and the Pharmaceutical Sciences) Edited by Alexander T. Florence and Juergen Siepmann, CRC Press, 2016. ISBN-10: 142006570X).

In some embodiments, the compositions described herein are formulated as a solution, sprayable solution, spray, gel, or a moderate to highly viscous solution. In some embodiments, the composition is a solution with a viscosity in the range of about 0.8-20 cP at standard temperature and pressure (“STP”). In some embodiments the composition is a light gel, for example, a low-viscosity gel or a sprayable gel. Alternatively, the composition is a high-viscosity gel. Low viscosity gels are, for example, gels having a dynamic viscosity in the range of about 400-4000 cP at STP. High viscosity gels are, for example, gels having a dynamic viscosity of at least 4000 cP at STP.

In some embodiments, a composition described herein is a liquid formulation. In some embodiments, a composition described herein is a sprayable liquid formulation. In some embodiments, the liquid formulation is applied topically to the subject. In some embodiments, a liquid formulation is delivered to the subject by an aerosol delivery system.

In some embodiments, a composition described herein is a spray formulation.

### Treatment Methods and Uses

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compositions or formulations described herein may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The terms “effective amount,” “pharmaceutically effective amount” and “therapeutically effective amount” refer to an amount that may be effective to elicit the desired biological or medical response, including the amount of a compound that, when administered to a subject for treating a disease, is sufficient to effect such treatment for the disease. The effective amount will vary depending on the compound, the disease and its severity and the age, weight, etc., of the subject to be treated. The effective amount can include a range of amounts. Further, an effective amount includes amounts of an agent which are effective when combined with other agents.

The compositions and formulations disclosed herein are useful for the treatment of a skin disorder. In some embodiments, the compositions and formulations disclosed herein are useful for the treatment of an inflammatory skin disorder. In some embodiments, the compositions and formulations disclosed herein are useful for the treatment of dermatosis.

Some embodiments provide for a method of treating a corticosteroid responsive dermatosis in a subject in need thereof comprising administering a composition or formulation as described herein. Some embodiments provide for a method of treating psoriasis in a subject in need thereof comprising administering a composition or formulation as described herein.

### Kits

Provided herein are also kits that include a composition or formulation of the disclosure and suitable packaging. In one embodiment, a kit further includes instructions for use. In one aspect, a kit includes a composition or formulation of the disclosure and a label and/or instructions for use of the compounds in the treatment of the indications, including the diseases or conditions, described herein. In one embodiment, a kit further includes instructions as to the pharmacological similarity or bioequivalence between the composition or formulation of the disclosure and a reference composition or formulation. Provided herein are also articles of manufacture that include a composition or formulation of the disclosure in a suitable container. The container may be a bottle, vial, sachet, manually-activated or powered aerosol or spray dispenser, a manually-activated or powered pump, or a manually-activated, powered or pressurized foam dispenser.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Preparation of Desoximetasone Formulations

The formulations summarized in Tables 1 to 7 were prepared as follows.

All materials were purchased from VWR and Gattefosse. A desired weight of desoximetasone was weighed in a glass media bottle. Isopropyl alcohol was then weighed and transferred into the media bottle. The media bottle was capped and the contents sonicated at room temperature until the desoximetasone fully dissolved. The remaining excipients were then added to the isopropanol and desoximetasone solution. The resulting mixture was then briefly mixed.

TABLE 1

| | Formulation name | | | | | | | | | |
|---|---|---|---|---|---|---|---|---|---|---|
| | TrDsx1 | TrDsx2 | TrDsx3 | TrDsx4 | TrDsx5 | TrDsx6 | TrDsx7 | TrDsx8 | TrDsx9 | TrDsx10 |
| Ingredient | wt/wt % | wt/wt % | wt/wt % | wt/wt % | wt/wt % | wt/wt % | wt/wt % | wt/wt % | wt/wt % | wt/wt % |
| Desoximetasone | 0.25 | 0.25 | 0.25 | 0.25 | 0.25 | 0.25 | 0.25 | 0.25 | 0.25 | 0.25 |
| Isopropyl alcohol | 18.00 | 45.00 | 18.00 | 45.00 | 18.00 | 75.00 | 23.40 | 23.40 | 23.40 | 23.40 |
| Sorbitan sesquioleate | | | | | | | 0.90 | | | |
| Glyceryl monooleate | | | | | | | | 0.90 | 0.90 | 0.90 |
| Isopropyl myristate | | | | | | | 31.38 | 8.00 | 8.00 | 8.00 |
| Isopropyl palmitate | 65.85 | 38.85 | 65.85 | 38.85 | 29.85 | 8.85 | | 7.00 | 7.00 | |
| Transcutol | | | | | | | | 16.37 | | 23.40 |
| Oleic acid | | | | | | | | | 16.37 | |
| Mineral oil | 15.00 | 15.00 | 15.00 | 15.00 | 51.00 | 15.00 | 44.02 | 44.03 | 44.03 | 44.00 |
| L-Menthol | | | | | | | 0.05 | 0.05 | 0.05 | 0.05 |
| Sodium lauryl sulfate | 0.90 | 0.90 | | | | | | | | |
| Tween 80 | | | 0.90 | 0.90 | 0.90 | 0.90 | | | | |

TABLE 2

| | Formulation name | | | | | | | | | |
|---|---|---|---|---|---|---|---|---|---|---|
| | TrDsx11 | TrDsx12 | TrDsx13 | TrDsx14 | TrDsx15 | TrDsx16 | TrDsx17 | TrDsx18 | TrDsx19 | TrDsx20 |
| Ingredient | wt/wt % | wt/wt % | wt/wt % | wt/wt % | wt/wt % | wt/wt % | wt/wt % | wt/wt % | wt/wt % | wt/wt % |
| Desoximetasone | 0.25 | 0.25 | 0.25 | 0.25 | 0.25 | | | 0.25 | 0.25 | 0.25 |
| Isopropyl alcohol | 23.40 | 24.20 | 24.20 | 65.80 | 65.80 | 66.10 | 66.10 | | | 49.70 |
| Sorbitan | | | | | | | | 59.70 | 54.70 | |
| sesquioleate | | | | | | | | | | |
| Glyceryl | 0.90 | 0.90 | 0.90 | 0.90 | 0.90 | 0.90 | 0.90 | 2.00 | 2.00 | 2.00 |
| monooleate | | | | | | | | | | |
| Isopropyl | 8.00 | 8.00 | 8.00 | 8.00 | 8.00 | 8.00 | 8.00 | 8.00 | 8.00 | 8.00 |
| myristate | | | | | | | | | | |
| Transcutol | | 5.00 | 10.00 | 25.00 | | 25.00 | | 25.00 | 25.00 | 25.00 |
| Oleic acid | 23.40 | | | | 25.00 | | 25.00 | | | |
| Mineral oil | 44.00 | 61.60 | 56.60 | | | | | 5.00 | 10.00 | 15.00 |
| L-Menthol | 0.05 | 0.05 | 0.05 | 0.05 | 0.05 | | | 0.05 | 0.05 | 0.05 |

TABLE 3

| | Formulation name | | | | | | | | | | |
|---|---|---|---|---|---|---|---|---|---|---|---|
| | TrDsx21 | TrDsx22 | TrDsx23 | TrDsx24 | TrDsx25 | TrDsx26 | TrDsx27 | TrDsx28 | TrDsx29 | TrDsx30 | TrDsx31 |
| Ingredient | wt/wt % | wt/wt % | wt/wt % | wt/wt % | wt/wt % | wt/wt % | wt/wt % | wt/wt % | wt/wt % | wt/wt % | wt/wt % |
| Desoximetasone | 0.25 | 0.25 | 0.25 | 0.25 | 0.25 | 0.25 | 0.25 | 0.25 | 0.25 | 0.25 | 0.25 |
| Isopropyl alcohol | 44.70 | 39.70 | 34.70 | 29.70 | 60.80 | 55.80 | 50.80 | 45.80 | 40.80 | 35.80 | 30.80 |
| Glyceryl | 2.00 | 2.00 | 2.00 | 2.00 | 0.90 | 0.90 | 0.90 | 0.90 | 0.90 | 0.90 | 0.90 |
| monooleate | | | | | | | | | | | |
| Isopropyl | 8.00 | 8.00 | 8.00 | 8.00 | 8.00 | 8.00 | 8.00 | 8.00 | 8.00 | 8.00 | 8.00 |
| myristate | | | | | | | | | | | |
| Transcutol | 25.00 | 25.00 | 25.00 | 25.00 | | | | | | | |
| Oleic acid | | | | | 25.00 | 25.00 | 25.00 | 25.00 | 25.00 | 25.00 | 25.00 |
| Mineral oil | 20.00 | 25.00 | 30.00 | 35.00 | 5.00 | 10.00 | 15.00 | 20.00 | 25.00 | 30.00 | 35.00 |
| L-Menthol | 0.05 | 0.05 | 0.05 | 0.05 | 0.05 | 0.05 | 0.05 | 0.05 | 0.05 | 0.05 | 0.05 |

TABLE 4

| | Formulation name | | | | | | |
|---|---|---|---|---|---|---|---|
| | TrDsx32 | TrDsx33 | TrDsx34 | TrDsx35 | TrDsx36 | TrDsx37 | TrDsx38 |
| Ingredient | wt/wt % | wt/wt % | wt/wt % | wt/wt % | wt/wt % | wt/wt % | wt/wt % |
| Desoximetasone | 0.25 | 0.25 | 0.25 | 0.25 | 0.25 | 0.25 | 0.25 |
| Isopropyl alcohol | 23.40 | 23.40 | 23.40 | 23.40 | 23.40 | 23.40 | 23.40 |
| Glyceryl monooleate | 0.90 | 0.90 | 0.90 | 0.90 | 0.90 | 0.90 | 0.90 |
| Isopropyl myristate | 8.00 | 8.00 | 8.00 | 8.00 | 8.00 | 8.00 | 8.00 |
| Propylene glycol | 23.40 | | | | | | |
| Butylene dioxide | | 23.40 | | | | | |
| Hexylene glycol | | | 23.40 | | | | |
| Dimethyl sulfoxide | | | | 23.40 | | | |
| PEG400 | | | | | 23.40 | | |
| Soybean oil | | | | | | 23.40 | |
| Caprylic/capric | | | | | | | 23.40 |
| triglyceride/GTCC | | | | | | | |
| Corn oil | | | | | | | |
| Glycerol | | | | | | | |
| Lauryl lactate | | | | | | | |
| Labrasol | | | | | | | |
| PEG40 castor oil | | | | | | | |
| Sorbitol | | | | | | | |
| Mineral oil | 44.00 | 44.00 | 44.00 | 44.00 | 44.00 | 44.00 | 44.00 |
| L-Menthol | 0.05 | 0.05 | 0.05 | 0.05 | 0.05 | 0.05 | 0.05 |

| | Formulation name | | | | | |
|---|---|---|---|---|---|---|
| | TrDsx39 | TrDsx40 | TrDsx41 | TrDsx42 | TrDsx43 | TrDsx44 |
| Ingredient | wt/wt % | wt/wt % | wt/wt % | wt/wt % | wt/wt % | wt/wt % |
| Desoximetasone | 0.25 | 0.25 | 0.25 | 0.25 | 0.25 | 0.25 |
| Isopropyl alcohol | 23.40 | 23.40 | 23.40 | 23.40 | 23.40 | 23.40 |
| Glyceryl monooleate | 0.90 | 0.90 | 0.90 | 0.90 | 0.90 | 0.90 |
| Isopropyl myristate | 8.00 | 8.00 | 8.00 | 8.00 | 8.00 | 8.00 |
| Propylene glycol | | | | | | |
| Butylene dioxide | | | | | | |
| Hexylene glycol | | | | | | |
| Dimethyl sulfoxide | | | | | | |
| PEG400 | | | | | | |
| Soybean oil | | | | | | |
| Caprylic/capric | | | | | | |
| triglyceride/GTCC | | | | | | |
| Corn oil | 23.40 | | | | | |
| Glycerol | | 23.40 | | | | |
| Lauryl lactate | | | 23.40 | | | |
| Labrasol | | | | 23.40 | | |
| PEG40 castor oil | | | | | 23.40 | |
| Sorbitol | | | | | | 23.40 |
| Mineral oil | 44.00 | 44.00 | 44.00 | 44.00 | 44.00 | 44.00 |
| L-Menthol | 0.05 | 0.05 | 0.05 | 0.05 | 0.05 | 0.05 |

TABLE 5

| | Formulation name | | | | |
|---|---|---|---|---|---|
| | TrDsx60 | TrDsx61 | TrDsx62 | TrDsx63 | TrDsx64 |
| Ingredient | wt/wt % | wt/wt % | wt/wt % | wt/wt % | wt/wt % |
| Desoximetasone | 0.25 | 0.25 | 0.25 | 0.25 | 0.25 |
| Isopropyl alcohol | 23.40 | 23.40 | 23.40 | 22.99 | 23.10 |
| Glyceryl monooleate | 2.00 | 0.90 | 2.00 | 2.95 | 1.97 |
| Isopropyl myristate | 8.00 | 8.00 | 8.00 | 7.86 | 7.90 |
| Isopropyl palmitate | | 7.50 | 7.50 | 7.37 | |
| Soybean oil | 22.15 | 15.90 | 14.65 | 14.39 | 23.15 |
| Mineral oil | 44.00 | 44.00 | 44.00 | 43.22 | 43.44 |
| L-Menthol | 0.20 | 0.05 | 0.20 | 0.98 | 0.20 |

TABLE 6

| | Formulation name | | | | |
|---|---|---|---|---|---|
| | TrDsx60 | TrDsx61 | TrDsx62 | TrDsx63 | TrDsx64 |
| Ingredient | wt/wt % | wt/wt % | wt/wt % | wt/wt % | wt/wt % |
| Desoximetasone | 0.25 | 0.25 | 0.25 | 0.25 | 0.25 |
| Isopropyl alcohol | 23.40 | 23.40 | 23.40 | 22.99 | 23.10 |
| Glyceryl monooleate | 2.00 | 0.90 | 2.00 | 2.95 | 1.97 |
| Isopropyl myristate | 8.00 | 8.00 | 8.00 | 7.86 | 7.90 |
| Isopropyl palmitate | | 7.50 | 7.50 | 7.37 | |
| Soybean oil | 22.15 | 15.90 | 14.65 | 14.39 | 23.15 |
| Mineral oil | 44.00 | 44.00 | 44.00 | 43.22 | 43.44 |
| L-Menthol | 0.20 | 0.05 | 0.20 | 0.98 | 0.20 |

TABLE 7

| | Formulation name | | | | | | | |
|---|---|---|---|---|---|---|---|---|
| | TrDsx70 | TrDsx71 | TrDsx72 | TrDsx73 | TrDsx74 | TrDsx82 | TrDsx83 | TrDsx84 |
| Ingredient | wt/wt % | wt/wt % | wt/wt % | wt/wt % | wt/wt % | wt/wt % | wt/wt % | wt/wt % |
| Desoximetasone | 0.25 | 0.25 | 0.25 | 0.25 | 0.25 | 0.25 | 0.25 | 0.25 |
| Isopropyl alcohol | 23.40 | 23.40 | 23.40 | 23.40 | 23.40 | 23.40 | 23.40 | 23.40 |
| Glyceryl monooleate | 2.00 | | 2.00 | 2.00 | 2.00 | 2.00 | 2.00 | 0.90 |
| Isopropyl palmitate | 8.00 | 8.00 | 7.15 | 5.30 | | 8.00 | | |
| Soybean oil | 22.15 | 22.65 | 15.00 | 22.15 | 22.15 | | | |
| Mineral oil | 44.00 | 44.00 | 44.00 | 42.70 | 44.00 | 44.00 | 44.00 | 44.00 |
| L-Menthol | 0.20 | 0.20 | 0.20 | 0.20 | 0.20 | 0.20 | 0.20 | 0.05 |
| Lauryl lactate | | 1.50 | | | | | | |
| Caprylic/capric | | | 8.00 | 4.00 | 8.00 | 22.15 | 30.15 | 31.40 |
| triglyceride/GTCC | | | | | | | | |

### Example 2: Stability Studies

Stability studies of formulations “TrDsx37” and “TrDsx62” (described in Tables 4 and 5) were performed as follows. The formulations were placed in duplicate, in borosilicate glass vial (2 ml volume). The formulations were studied at accelerated temperature (40±2° C.) and room temperature (25±2° C.) for 4 weeks. At each of 1,2,3 and 4 weeks each vial was removed and analyzed by stability-indicating HPLC method for degradation products.

Table 8 below summarizes the wt % of desoximetasone at 40±2° C. at 0, 2, 3, and 4 weeks.

TABLE 8

| Week | TrDsx37 | TrDsx62 |
|---|---|---|
| 0 | 0.24% | 0.24% |
| 2 | 0.24% | 0.23% |
| 3 | 0.24% | 0.23% |
| 4 | 0.24% | 0.23% |

Stability studies of formulations “TrDsx37” and “TrDsx73” (described in Tables 4 and 7) and to each of these two formulations into which 0.1% w/w of the common antioxidant butylated hydroxytoluene (BHT) had been added were performed as follows. The formulations were placed in duplicate, in borosilicate glass vial (2 ml volume). The formulations were studied at accelerated temperature (40±2° C.) and room temperature (25±2° C.) for 7 weeks. At each of 5 and 7 weeks a vial was removed and analyzed by stability-indicating HPLC method for degradation products.

Table 9 below summarizes the assays for desoximetasone in these samples at 40±2° C. at 5 and 7 weeks, and at 25±2° C. for 7 weeks, expressed both on w/w (mg/g) and % potency (concentration of desoximetasone relative to that measured in the same formulation at time zero).

TABLE 9

| | Sampling | | Assay: | Assay: |
|---|---|---|---|---|
| Formulation | Time | Storage Condition | mg/g | % Potency |
| TrDsx37 #1 | 5 weeks | 40° C. | 2.54 | 100.2 |
| TrDsx37 w/ BHT#1 | 5 weeks | 40° C. | 2.58 | 102.5 |
| TrDsx73 #1 | 5 weeks | 40° C. | 2.46 | 97.9 |
| TrDsx73 w/BHT #1 | 5 weeks | 40° C. | 2.51 | 101.2 |
| TrDsx37 #2 | 7 weeks | 40° C. | 2.51 | 99.0 |
| TrDsx37 w/ BHT#2 | 7 weeks | 40° C. | 2.50 | 99.3 |
| TrDsx73 #2 | 7 weeks | 40° C. | 2.43 | 96.8 |
| TrDsx73 w/BHT #2 | 7 weeks | 40° C. | 2.48 | 100.0 |
| TrDsx37 #5 | 7 weeks | 25° C. (Spray | 2.50 | 98.5 |
| | | Cap) | | |
| TrDsx37 w/BHT #5 | 7 weeks | 25° C. (Spray | 2.46 | 97.9 |
| | | Cap) | | |
| TrDsx73 #5 | 7 weeks | 25° C. (Spray | 2.45 | 97.3 |
| | | Cap) | | |
| TrDsx73 w/BHT #5 | 7 weeks | 25° C. (Spray | 2.46 | 99.5 |
| | | Cap) | | |
| TrDsx37 #6 | 7 weeks | 25° C. | 2.52 | 99.6 |
| TrDsx37 w/BHT #6 | 7 weeks | 25° C. | 2.46 | 97.9 |
| TrDsx73 #6 | 7 weeks | 25° C. | 2.47 | 98.1 |
| TrDsx73 w/BHT #6 | 7 weeks | 25° C. | 2.48 | 100.0 |

The data demonstrate that compositions comprising isopropyl palmitate surprisingly exhibit notable stabilities. Such compositions comprising low concentrations of the known MPE™ isopropyl myristate or from which isopropyl myristate is completely absent form stable liquid formulations of desoximetasone.

### Example 3: Transdermal Flux Tests

Formulations as described in Example 1 were analyzed via a skin permeation test. “Control,” a commercially available formulation, was used as a comparative formulation. Control is believed to be composed of the following: about 0.25 wt % desoximetasone, glyceryl oleate, about 23 wt % isopropyl alcohol, about 32 wt % isopropyl myristate, less than 1 wt % L-menthol, and mineral oil.

To assay transdermal flux of desoximetasone from the formulations across the skin barrier, traditional Franz diffusion cells were used. Franz diffusion cells (“FDCs”) were assembled by placing a piece of human cadaver skin, stratum corneum side facing up, between the receptor and donor chambers of the diffusion cell and clamping the chambers together with a pinch clamp. Human cadaver skin is chosen as the substrate as it mimics in vivo conditions. The receptor chambers were filled with phosphate buffered saline solution at pH 7.4 with 0.01 wt % NaN₃ added as a preservative and 2 wt % hydroxypropyl-β-cylclodextrin added as a solubilizing agent to ensure sink conditions in the receptor fluid throughout the experiment. In addition, a stir bar was added to each receptor chamber. Care was taken to ensure that any air bubbles underneath the skin were removed prior to beginning the flux study. The assembled FDCs were then placed in a stirring dry bath heater. The receptor chambers were continually stirred throughout the experiment and the temperature maintained at a constant 32° C.

After the cells were assembled, a tritiated water prescreening was performed to test the integrity of the skin piece in each FDC. This tritiated water prescreening consisted of adding an aliquot of tritiated water to the skin, allowing it to sit on the skin surface for 5 minutes after which it was removed using a pipette. A 300 μl aliquot was then collected from the receptor chamber after a further hour of incubation and placed into a well in a microtiter plate. 600 μL of scintillation cocktail (Ultima Gold from Perkin Elmer) was added to each sample aliquot in the microtiter plate and the tritium (³H) content of each sample aliquot was then measured using a liquid scintillation counter (PerkinElmer MicroBeta TriLux 1450). FDCs demonstrating anomalously high tritiated water flux values were discarded and the remaining cells were ranked according to the magnitudes of the measured tritiated water flux values. Test formulations were then assigned to batches of FDCs such that the replicates for each test formulation were applied to skin pieces with nearly equivalent average tritiated water flux values.

After the tritiated water prescreening was complete, 6-fold replicates of each test formulation were applied to the skin using a positive displacement pipette. Formulations were then spread across the skin surface with a glass rod. After dosing was complete, samples were then taken from the receptor chamber at regular intervals over a period of 24 hours with fresh receptor solution being introduced to replace the removed sample aliquot. At the end of the 24 hour period, the surface of the skin was wiped clean with a water/ethanol soaked Kimwipe and tapped dry. The skin was then tape stripped three times, with the tape strips being discarded (the active agent in such tape strips being considered only superficially absorbed). The remaining skin was then separated into epidermal and dermal compartments, using mild heating if necessary. The epidermal and dermal pieces were placed into glass vials and an extraction solvent (dimethyl sulfoxide) was added to each vial. The vials were then incubated for 24 hours at 40° C. with gentle agitation. After 24 hours of incubation, samples were collected. All of the receptor and extraction samples were analyzed via HPLC analysis for desoximetasone content. The reported flux values were calculated from the cumulative amounts of the drug in the receiver compartment versus time and the cumulative amounts in the epidermal and dermal compartments.

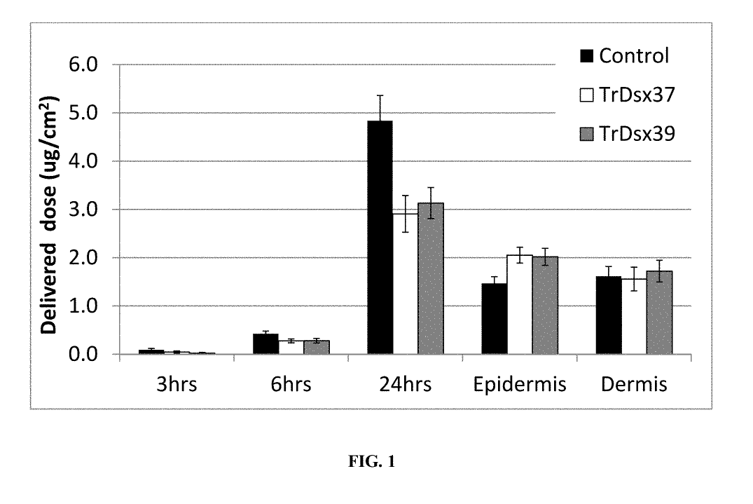

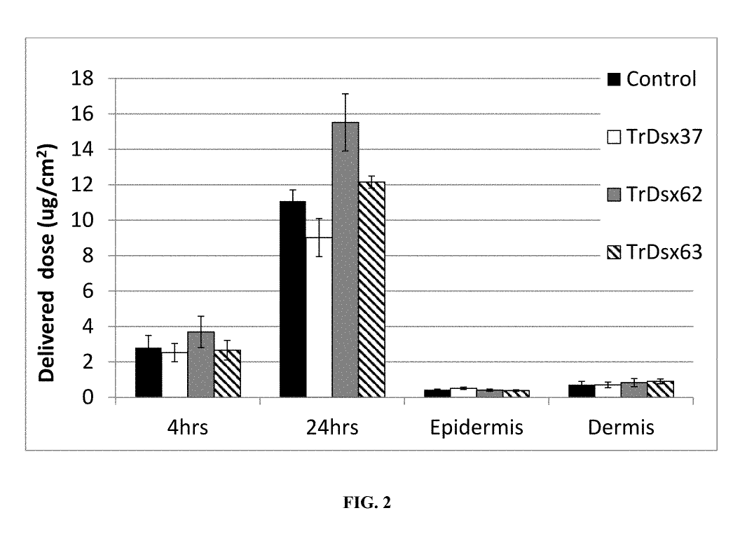

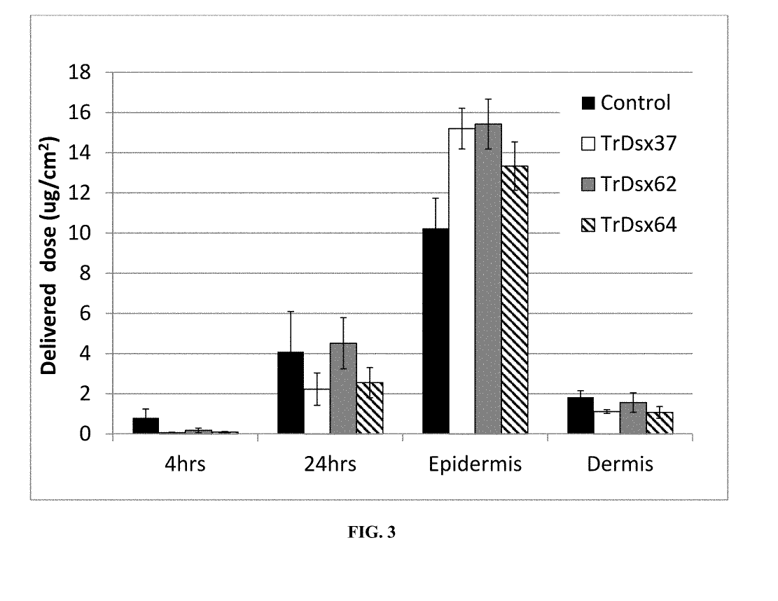

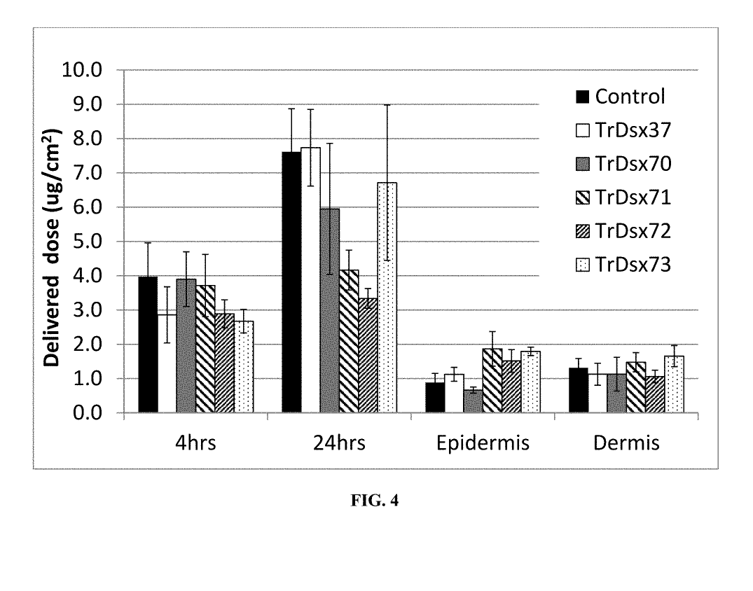

The results of the skin delivery and permeation measurements are summarized in FIG. 1 to FIG. 4. The data demonstrate that compositions comprising isopropyl palmitate, even at low concentrations, surprisingly exhibit remarkable skin permeation and retention. Such compositions comprising low concentrations of the known MPE™ isopropyl myristate or from which isopropyl myristate is completely absent evidence desoximetasone permeation and delivery closely comparable to those from the Control formulation that comprises more than 30° % w/w of isopropyl myristate. It is contemplated that the skin permeation and delivery of other active agents could benefit from incorporation of isopropyl palmitate.

### Example 4: Spray Pattern Tests

The dispense characteristics of formulations of the disclosure, both formulation amounts dispensed and spray patterns, were assessed using two spray pumps, one (“RT-1”) provided with a commercial Control and a second (“NT-”) of similar but not necessarily identical design. Results from formulations TrDsx37 and TrDsx62 were compared to those of “Control,” the commercially available formulation using both RT-1 and NT-1, as summarized by Table 10. Fifty sprays from each formulation-container configuration were tested. Total dispensed weights from sprays S1-S12, S30, S40, and S50 were collected and measured.

TABLE 10

| | Bottle | S1 | S2 | S3 | S4 | S5 | S6 | S7 | S8 | S9 | S10 | S11 | S12 | S30 | S40 | S50 | Avg | StdDev |
|---|---|---|---|---|---|---|---|---|---|---|---|---|---|---|---|---|---|---|
| Formln | Type | mg | mg | mg | mg | mg | mg | mg | mg | mg | mg | mg | mg | mg | mg | mg | mg | mg |
| Control | RT-1 | 117.0 | 119.0 | 112.3 | 118.9 | 119.3 | 118.5 | 118.2 | 117.6 | 118.7 | 118.0 | 118.5 | 118.6 | 119.1 | 117.6 | 118.0 | 118.0 | 1.7 |
| Control | RT-1 | 123.3 | 123.7 | 123.7 | 123.4 | 124.0 | 122.8 | 122.3 | 123.8 | 123.1 | 122.8 | 123.7 | 119.3 | 124.3 | 123.7 | 124.4 | 123.2 | 1.2 |
| Control | NT-1 | 170.6 | 170.7 | 183.9 | 185.1 | 181.3 | 170.4 | 182.2 | 170.9 | 168.7 | 168.7 | 168.5 | 171.6 | 160.0 | 166.5 | 167.5 | 172.4 | 7.3 |
| Control | NT-1 | 147.7 | 146.6 | 151.3 | 137.9 | 146.8 | 152.9 | 132.2 | 150.8 | 147.4 | 147.8 | 137.2 | 135.6 | 152.1 | 137.4 | 154.4 | 145.2 | 7.2 |
| TrDsx37 | NT-1 | 178.6 | 177.9 | 181.2 | 175.3 | 176.2 | 178.9 | 183.5 | 181.4 | 180.1 | 177.4 | 174.2 | 180.8 | 178.1 | 173.1 | 173.8 | 178.0 | 3.1 |
| TrDsx62 | NT-1 | 165.8 | 159.0 | 163.2 | 162.0 | 161.6 | 160.6 | 165.8 | 156.0 | 170.0 | 168.5 | 163.1 | 158.7 | 142.6 | 163.4 | 162.9 | 161.5 | 6.4 |

Additional formulations as summarized in Table 11 were tested for spray actuation content in eight replicates per formulation using a different container closure system, NT-2, and results were compared to those of the Control formulation.

TABLE 11

| | Control | TrDsx37 | TrDsx73 | TrDsx74 |
|---|---|---|---|---|
| Replicate | mg | mg | mg | mg |
| 1 | 117.1 | 119.0 | 118.9 | 121.6 |
| 2 | 119.2 | 118.3 | 119.9 | 122.3 |
| 3 | 118.6 | 117.0 | 117.6 | 124.1 |
| 4 | 117.4 | 118.9 | 119.2 | 123.1 |
| 5 | 119.2 | 118.2 | 121.9 | 122.3 |
| 6 | 119.5 | 118.0 | 122.8 | 121.3 |
| 7 | 119.4 | 117.9 | 119.6 | 120.7 |
| 8 | 120.3 | 118.2 | 117.4 | 119.6 |
| Average | 118.8 | 118.2 | 119.7 | 121.9 |

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All publications, patent applications, patents, and other references mentioned herein are expressly incorporated by reference in their entirety, to the same extent as if each were incorporated by reference individually. In case of conflict, the present specification, including definitions, will control.

1. A composition comprising:
(a) about 0.01 wt % to about 2.5 wt % desoximetasone;
(b) isopropyl palmitate;
(c) about 10 wt % to about 80 wt % C₂₋₄ alcohol;
(d) about 3 wt % to about 75 wt % of an oleaginous vehicle selected from mineral oil, soybean oil, caprylic/capric triglycerides, and a combination thereof; and
(e) about 0.01 wt % to about 10 wt % of glyceryl monooleate.

2. The composition of claim 1, wherein isopropyl palmitate is present in an amount of about 2 wt % to about 12 wt %.

3.-4. (canceled)

6. The composition of claim 1, wherein the C₂₋₄ alcohol is isopropyl alcohol.

7.-12. (canceled)

13. The composition of claim 1, further comprising 0.001 wt % to about 5% menthol.

14.-15. (canceled)

16. The composition of claim 1, wherein the composition is a sprayable liquid formulation.

17. A method of treating a corticosteroid responsive dermatosis in a subject in need thereof comprising administering the liquid formulation of claim 16.

18. The method of claim 17, wherein the liquid formulation is applied topically to the subject.

19. (canceled)

20. A method for preparing a composition of claim 1 comprising:
(a) dissolving the desoximetasone in the C₂₋₄ alcohol; and
(b) adding the isopropyl palmitate, the oleaginous vehicle, and the glyceryl monooleate.

21. A topical composition comprising:
(a) about 0.01 wt % to about 2.5 wt % of a corticosteroid;
(b) an ester of a C₂₋₄ alcohol and a fatty acid;
(c) about 10 wt % to about 80 wt % C₂₋₄ alcohol;
(d) about 3 wt % to about 75 wt % of an oleaginous vehicle selected from mineral oil, soybean oil, caprylic/capric triglycerides, and a combination thereof; and
(e) about 0.01 wt % to about 10 wt % of a glyceryl ester of a fatty acid;
wherein the ester of a C₂₋₄ alcohol and a fatty acid is not isopropyl myristate.

22. The composition of claim 21, wherein the corticosteroid is desoximetasone.

23. The composition of claim 21, wherein the ester of a C₂₋₄ alcohol and a fatty acid is isopropyl palmitate present in an amount of about 2 wt % to about 12 wt %.

24.-25. (canceled)

26. The composition of claim 21, wherein the C₂₋₄ alcohol is isopropyl alcohol.

27.-31. (canceled)

32. A topical composition comprising an active agent and an ester of a C₂₋₄ alcohol and a fatty acid, wherein the active agent is delivered to the dermis at a dose of greater than about 1 μg of active agent per cm² area of skin in up to 24 hours.

33. The topical composition of claim 32, wherein the active agent is desoximetasone.

34. The topical composition of claim 32, wherein the ester of a C₂₋₄ alcohol and a fatty acid is not isopropyl myristate.

35. The topical composition of claim 32, wherein the ester of a C₂₋₄ alcohol and a fatty acid is isopropyl palmitate.

36. (canceled)

37. A topical formulation comprising desoximetasone, wherein the topical formulation has a similar profile of delivery of desoximetasone to a dermal tissue of a subject as a target formulation;
wherein the target formulation comprises about 0.25 wt % desoximetasone, glyceryl monoleate, about 23 wt % isopropyl alcohol, about 32 wt % isopropyl myristate, less than about 1 wt % L-menthol, and mineral oil; and
wherein the topical formulation does not comprise isopropyl myristate.

38.-39. (canceled)

40. The topical formulation of claim 37, wherein the topical formulation comprises an ester of a C₂₋₄ alcohol and a fatty acid, wherein desoximetasone is delivered to the dermis at a dose of greater than about 1 μg of desoximetasone per cm² area of skin in up to 24 hours.

41. A method of identifying a topical formulation of claim 37, said method comprising:
(a) measuring delivery of desoximetasone of the target formulation;
(b) preparing a test composition comprising desoximetasone and one or more excipients of the target formulation;
(c) measuring delivery of desoximetasone of the test composition;
(d) comparing the delivery of desoximetasone of the test composition with the delivery of desoximetasone of the target formulation; and
(e) modifying one or more excipients of the test composition.

42. (canceled)

43. A composition comprising:
(a) about 0.01 wt % to about 2.5 wt % of a corticosteroid;
(b) about 10% to 80 wt % C₂₋₄ alcohol;
(c) about 0.01 wt % to about 10 wt % of glyceryl monooleate;
(d) about 3 wt % to about 80 wt % of an oleaginous vehicle selected from mineral oil, soybean oil, caprylic/capric triglycerides, and a combination thereof; and
(e) an ester of a C₂₋₄ alcohol and a fatty acid; wherein the ester of a C₂₋₄ alcohol and a fatty acid is not isopropyl myristate.

44. The composition of claim 43, wherein the ester of a C₂₋₄ alcohol and a fatty acid is isopropyl palmitate, and isopropyl palmitate is present in an amount of about 2 wt % to about 12 wt %.

45. (canceled)

46. The composition of claim 43, wherein the corticosteroid is desoximetasone present in an amount of about 0.2 wt % to about 0.5 wt %.

47.-58. (canceled)

59. The composition of claim 43, wherein the ester of a C₂₋₄ alcohol and a fatty acid is present in an amount of less than 10 wt %.

60.-61. (canceled)

62. A method of treating a corticosteroid responsive dermatosis in a subject in need thereof comprising administering the composition of claim 43, wherein the composition is a liquid formulation.

63.-64. (canceled)
